(19) United States
(12) Patent Application Publication
(10) Pub. No.: US 2010/0034766 A1
(43) Pub. Date: Feb. 11, 2010

(54) Malodor Counteracting Compositions

(75) Inventors: Thomas McGee, Nyack, NY (US); Felix Flachsmann, Duebendort (CH); Markus Gautschi, Zeiningen (CH); Philip Kraft, Duebendorf (CH); Richard P. Sgaramella, Hoboken, NJ (US); Kumar Venkateswara Vedantam, Paramus, NJ (US)

Correspondence Address: PARFOMAK, ANDREW N.;NORRIS MCLAUGHLIN & MARCUS PA, 875 THIRD AVE, 8TH FLOOR, NEW YORK, NY, 10022, US

(73) Assignee: Givaudan SA, Vemier (CH)

(21) Appl. No.: 12/444,752

(22) PCT Filed: Oct. 22, 2007
(86) PCT No.: PCT/CH07/00522

§ 371 Date: Apr. 22, 2009

Related U.S. Application Data
(60) Provisional application No. 60/862,622, filed on Oct. 24, 2006.

(51) Int. Cl. A61L 9/01; C07C 69/74; C07C 49/00

(52) U.S. Cl. 424/76.1; 560/126; 568/376

(57) ABSTRACT

A malodor counteracting composition comprising at least one compound of formula (A)

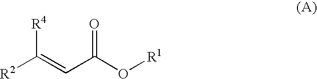

and at least one compound of formula (B)

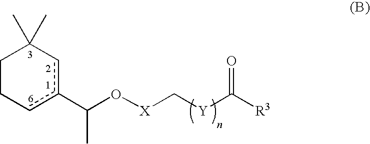

wherein n, X, Y and R¹ to R⁴ are as defined in the description.

The invention relates to malodor counteracting compositions. More particularly, the present invention relates to particular fragrance compositions containing two different classes of carboxylic acid esters.

Malodors are offensive odors, which are encountered in the air and on many substrates such as fabric, hard surfaces, skin and hair. They have either personal or environmental origin. For example sweat, urine, and feces malodors are personal in origin, whereas kitchen, gasoline, cooking, tobacco smoke, etc. malodors are of environmental origin. While personal malodors are easily deposited on fabric, hair, and skin, environmental malodors also have a propensity to deposit on these substrates. Combinations of personal and environmental malodors make up a composite malodor, which comprises many oil-soluble, water-soluble, and solid components that are highly volatile and are therefore easily perceived by humans.

Several approaches have been used to counteract malodors. These approaches include masking by superimposing on the malodor a pleasant stronger odor, cross-adaptation by blocking of the malodor olfactory receptors, suppression of the malodor by mixing with an ingredient that causes a negative deviation of Raoult's law, elimination of the malodor by chemical reaction, absorption of the malodor by a porous or cage-like structure, and avoidance of the formation of malodors by such routes as antimicrobials and enzyme inhibitors. Whereas all of these approaches are more or less effective there is still an ongoing demand for even more efficient products which may allow the use of lower concentrations of active compounds in an end product.

It has now been found that the combination of a known class of malodor counteractant molecules, as described for example in WO 02/051788 and U.S. Pat. No. 4,305,930, with a selected class of carboxylic acid esters results in very efficient malodor counteractant compositions. Surprisingly, the specific combination of the two classes of molecules, as described herein under, results in an unexpected synergistic increase in counteracting capabilities.

Accordingly, the present invention refers in one of its aspects to a composition comprising
a) at least one compound of formula (A)

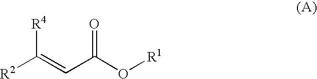

wherein
R¹ is linear or branched C₁-C₁₂ alkyl, e.g. ethyl and hexyl, linear or branched C₃-C₁₂ alkenyl, e.g. citronellyl, C₆-C₁₁ aryl, e.g. phenyl, C₇-C₁₂ arylalkyl, e.g. benzyl, or C₇-C₁₂ arylalkyl substituted with at least one O or N atom;
R² is linear or branched C₁-C₁₀ alkyl, e.g. methyl, ethyl and isopropyl, C₆-C₁₀ aryl, e.g. phenyl, C₇-C₁₀ alkoxyaryl, e.g. 4-methoxyphenyl, C₄-C₁₀ alkoxycarbonyl, e.g. hexyloxycarbonyl;
R⁴ is hydrogen or methyl; and
R² and R⁴ are either in the E or Z configuration with respect to the ester group; and
b) at least one compound of formula (B)

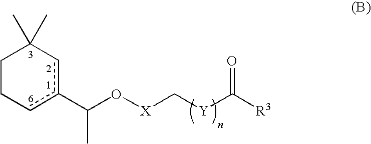

wherein
n is 0 or 1;
X is selected from the list of bivalent residues —C(CH₃)₂— and —C(O)—;
Y is selected from the list of bivalent residues —O—, —CH(CH₃)— and —CH₂—;
R³ is selected from the group consisting of C₁-C₃ alkyl such as methyl or ethyl,
C₂-C₃ alkenyl such as vinyl, C₃-C₅ cycloalkyl such as cyclopropyl or cyclobutyl,
C₁-C₃ alkoxy such as methoxy or ethoxy, and NR′R″ wherein R′ and R″ are independently of each other hydrogen, methyl or ethyl; and
the bonds between C-1 and C-2, and C-1 and C-6 are single bonds; or one of the bonds between C-1 and C-2, and C-1 and C-6 together with the dotted line represents a double bond.

In a preferred embodiment the compounds of formula (A) may be selected from citronellyl methylcrotonate (=3,7-dimethyloct-6-enyl 3-methylbut-2-enoate), geranyl crotonate, dihexyl fumarate, benzyl cinnamate, phenyl cinnamate and 2-ethyl-hexyl-para-methoxy-cinnamate (═Octyl methoxy cinnamate).

The compounds of formula (B) may be selected from the list of compounds given in Table 1 and Table 2.

TABLE 1
Compounds of formula (B) wherein n is 0.

| Comp. | X= | R³= | C-1 and C-2 | C-1 and C-6 |
|---|---|---|---|---|
| A | —C(CH₃)₂— | methyl | SB | SB |
| B | —C(O)— | methyl | SB | SB |
| C | —C(O)— | —O—C₂H₅ | SB | SB |
| D | —C(CH₃)₂— | —O—C₂H₅ | SB | SB |
| E | —C(CH₃)₂— | ethyl | SB | SB |

SB = single bond

TABLE 2
Compounds of formula (B) wherein n is 1.

| | | | | | C-1 |
|---|---|---|---|---|---|
| | | | | C-1 and | and |
| Comp. | X = | Y = | R³ = | C-2 | C-6 |
| 1 | —C(CH₃)₂— | —O— | ethyl | SB | SB |
| 2 | —C(O)— | —O— | ethyl | SB | SB |
| 3 | —C(CH₃)₂— | —O— | ethyl | DB | SB |
| 4 | —C(CH₃)₂— | —O— | cyclopropyl | SB | SB |
| 5 | —C(O)— | —O— | cyclopropyl | SB | SB |
| 6 | —C(CH₃)₂— | —O— | cyclopropyl | DB | SB |
| 7 | —C(CH₃)₂— | —O— | vinyl | SB | SB |
| 8 | —C(O)— | —O— | vinyl | SB | SB |
| 9 | —C(CH₃)₂— | —O— | vinyl | DB | SB |
| 10 | —C(CH₃)₂— | —CH₂— | methyl | SB | SB |
| 11 | —C(O)— | —CH₂— | methyl | SB | SB |
| 12 | —C(O)— | —CH₂— | methoxy | SB | SB |
| 13 | —C(O)— | —CH(CH₃)— | methyl | SB | SB |
| 14 | —C(O)— | —CH(CH₃)— | methoxy | SB | SB |
| 15 | —C(O)— | —CH(CH₃)— | —N(CH₃)₂ | SB | SB |
| 16 | —C(O)— | —CH₂— | ethoxy | SB | SB |

SB = single bond;
DB = double bond

The composition as hereinabove described preferably comprises a mixture of compound(s) of formula (A) and compound(s) of formula (B) in a ratio of from about 1:99 to about 99:1, preferably 90:10 to 50:50, e.g. 80:20 (A:B).

The composition of the present invention may further comprise other malodor counteractants such as malodor neutralizers and malodor absorbers. By “malodor neutralizer” is meant a material or a mixture thereof that reacts with malodor compounds such as certain amines, thiols, and short chain aliphatic acids. They may be preferably selected from aldehydes, such as alkyl aldehydes, benzaldehyde and vanillin; and cycloalkyl tertiary alcohols, such as 4-cyclohexyl-4-methyl-2-pentanone. By “malodor absorbers” is meant any material of large surface area capable of absorbing malodor. Such malodor absorbers include, for example, molecular sieves, such as zeolites, silicas, aluminosilicates, cyclodextrins, activated charcoal, clays, dried citrus pulp, cherry pit extract, corncob, and mixtures thereof.

The composition of the present invention may further comprise ingredients that retard the rate of build-up of malodor caused by bacterial breakdown, for example, antimicrobial agents and enzyme inhibitors. Such antimicrobial agents and enzyme inhibitors include, for example, metal salts such as zinc citrate, zinc oxide, zinc pyrethiones, and octopirox; organic acids, such as sorbic acid, benzoic acid, and their salts; parabens, such as methyl paraben, propyl paraben, butyl paraben, ethyl paraben, isopropyl paraben, isobutyl paraben, benzyl paraben, and their salts; alcohols, such as benzyl alcohol, phenyl ethyl alcohol; boric acid; 2,4,4′-trichloro-2-hydroxy-diphenyl ether (Triclosan™); phenolic compounds, such as phenol, 2-methyl phenol, 4-ethyl phenol; essential oils such as rosemary, thyme, lavender, eugenol, geranium, tea tree, clove, lemon grass, peppermint, or their active components such as anethole, thymol, eucalyptol, farnesol, menthol, limonene, methyl salicylate, salicylic acid, terpineol, nerolidol, geraniol, and mixtures thereof.

Optionally, the composition as defined above, i.e. a composition comprising at least one compound of formula (A) and at least one compound of formula (B), may be used in combination with known odorant molecules. Such molecules are, for example, described in “Perfume and Flavor Materials of Natural Origin”, S. Arctander, Ed., Elizabeth, N.J., 1960; “Perfume and Flavor Chemicals”, S. Arctander, Ed., Vol. I & II, Allured Publishing Corporation, Carol Stream, USA, 1994. Preferred are odorant molecules which are known as so called deodorant perfume components, as disclosed for example in U.S. Pat. No. 4,663,068 which are hereby incorporated by reference.

Auxiliary ingredients, such as solvents, dyes and antioxidants may also be added to the composition of the present invention in art-recognised quantities.

The solvents for use in the invention may be polar, such as ethanol, isopropanol, diethyleneglycol monoethyl ether, dipropylene glycol, diethyl phthalate, triethyl citrate, and triethylene glycols, or non-polar, such as isopropyl myristate and isoparaffinic hydrocarbons. Polar and non-polar solvents may be combined. The ratio of polar and non-polar solvents may be selected to provide the right properties for application and rate of release of the compounds of formula (A) and formula (B), as hereinabove described.

The compositions of the present invention may be added to a wide variety of consumer products, such as household products, personal care products and cosmetics, both perfumed and perfume-free.

Household products which may comprise a composition according to the invention include fabric washing powder and washing liquid, detergent, surface cleaner, including hard surface cleaner, air freshener, softener, bleach, fabric refresher and room spray, disinfection products, scourer and cat litter. The list of household products is given by way of illustration and is not to be regarded as being in any way limiting.

Personal care products and cosmetics which may comprise a composition according to the invention include lotion, e.g. after-shave lotion, shampoo, conditioner, styling spray, mousse, gel, hair wipe, hair spray, hair pomade, bath and shower gel, bath salt, hygiene product, deodorant, antiperspirant, vanishing crème, depilatory, talcum powder and catamenial. The list of personal care products and cosmetics is given by way of illustration and is not to be regarded as being in any way limiting.

Typically the products comprise from about 0.0001% to about 20% by weight, preferably about 0.001% to about 10% by weight, of at least one compound of formula (A) and at least one compound of formula (B) as hereinabove defined, based on the product. The effective amount depends upon the type of product into which the mixture is admixed. For example, if used in a fabric refresher it may be added to a fragrance composition at around 1% by weight which is then added to the product at around 0.1% by weight, i.e. the fabric refresher comprises about 0.001% by weight of the composition as hereinabove described. Or, in a liquid electrical air freshener composition it may be added at around 20% by weight based on the air freshener composition.

Accordingly, the present invention refers in a further aspect to a consumer product comprising an effective malodor-counteracting amount of a composition comprising at least one compound of formula (A) and at least one compound of formula (B).

Another aspect of the invention is a method of removing malodor from the air or from surfaces, comprising applying thereto an effective amount of a composition comprising at least one compound of formula (A) and at least one compound of formula (B) as hereinabove described.

In a further aspect the invention refers to a method of enhancing the malodor reduction properties of a consumer product, such as household products, personal care products and cosmetics, comprising admixing to the product at least one compound of formula (A) and at least one compound of formula (B) as hereinabove described.

Whereas some compounds falling within the definition of formula (B) are known as fragrances, others have never been described in literature.

Accordingly, the present invention refers in a further aspect to compounds of formula (B′)

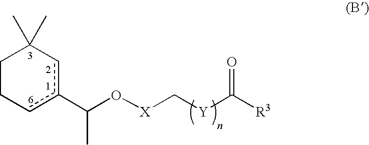

wherein
the bonds between C-1 and C-2, and C-1 and C-6 are single bonds; or one of the bonds between C-1 and C-2, and C-1 and C-6 together with the dotted line represents a double bond; and

### I) n is 1;

X is selected from the list of bivalent residues —C(CH₃)₂— and —C(O)—;
Y is selected from the list of bivalent residues —CH(CH₃)— and —CH₂—; and
R³ is selected from the group consisting of CH₃, OCH₃, OC₂H₅ and N(CH₃)₂;

### II) n is 0;

X is —C(CH₃)₂—;
R³ is selected from the group consisting of C₁-C₃ alkyl such as methyl or ethyl, C₂-C₃ alkenyl such as vinyl, C₃-C₅ cycloalkyl such as cyclopropyl or cyclobutyl, C₁-C₃ alkoxy such as methoxy or ethoxy, and NR′R″ wherein R′ and R″ are independently of each other hydrogen, methyl or ethyl; or

### III) n is 0;

X is —C(O)—; and
R³ is selected from the group consisting of C₂-C₃ alkyl such as ethyl or isopropyl, C₂-C₃ alkenyl such as vinyl, C₃-C₅ cycloalkyl such as cyclopropyl or cyclobutyl, methoxy, and NR′R″ wherein R′ and R″ are independently of each other hydrogen, methyl or ethy; with the proviso that 1-(3,3-dimethylcyclohexyl)ethyl methyl malonate is excluded.

The compounds of formula (B′) comprise at least one chiral centre and as such they may exist as a mixture of enantiomers and diastereomers, or they may be resolved as enantiomerically and diastereomerically pure forms. However, resolving stereoisomers adds to the complexity of manufacture and purification of these compounds and so it is preferred to use a compound of formula (B′) as a mixture of its stereoisomers simply for economic reasons. However, if it is desired to prepare pure stereoisomers, this may be achieved according to methodology known in the art. Accordingly the present invention refers in a further aspect to a compound of formula (B′) in the form of anyone of its isomers or a mixture thereof.

The compounds of formula (B′) wherein X is —C(O)— may be prepared via esterification of cyclademol (1-(3,3-dimethylcyclohexyl)ethanol) or its unsaturated analogs (I) and the appropriate acid/acyl chloride (II) as shown in Scheme 1 or may be prepared starting from cyclademol/unsaturated analogs (I) and the appropriated substituted succinic anhydride (III) as shown in Scheme 2, under conditions known to the person skilled in the art.

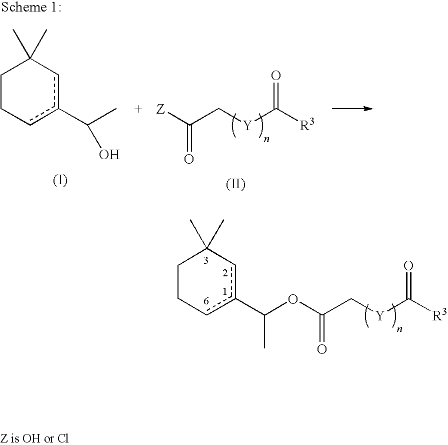

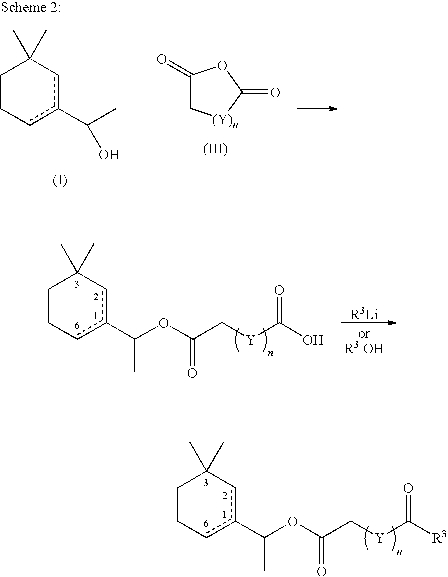

The compounds of formula (B′) wherein X is —C(CH₃)₂— may be prepared via 1,4-addition starting from cyclademol/unsaturated analoges (I) and a methylcrotonate (IV wherein for R³=alkoxy) or an isopropylidene ketone (IV wherein R³=alkyl, alkenyl, cycloalkyl) as shown in Scheme 3.

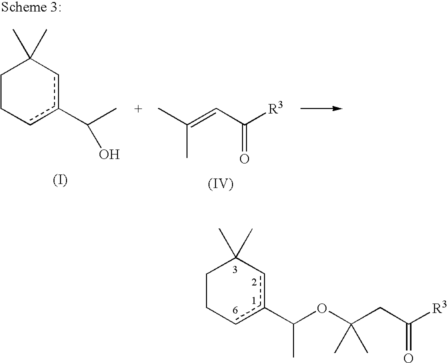

Compounds of formula (B′) wherein the bonds between C-1 and C-2, and C-1 and C-6 are single bonds and Y is —CH(CH₃)— or —CH₂— may also be prepared according to the general procedure as shown in Scheme 4.

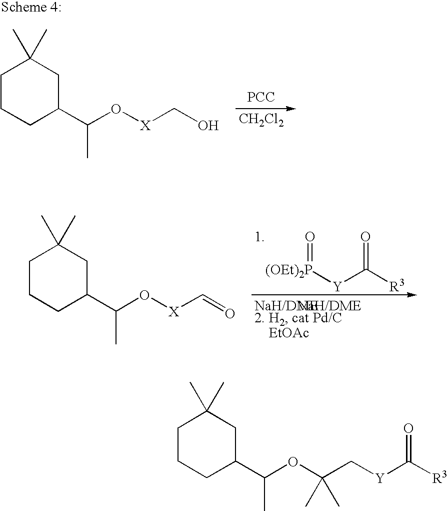

In the schemes 1 to 4 X, Y, n, and R³ do have the meaning as indicated in formula (B) above. The reactions represented in the schemes 1 to 3 are conventional reactions, the specific conditions of which are described in detail in the examples.

The invention is now further described with reference to the following non-limiting examples. These examples are of the purpose of illustration only and it is understood that variations and modifications can be made by one skilled in the art.

In this application, the term “%” or “percent” shall mean percent by weight, unless the context clearly indicates otherwise.

### EXAMPLE 1

### Determination of the Malodor Reduction Potential

A 1 liter glass headspace collection jar had placed inside it a 25 ml stoppered glass container which contained 0.5 g of a test compound/mixture of compounds as indicated in Table 3. 10 ul of hexyl amine as a representative malodor was injected into the headspace jar. This was left for 15 minutes at 25° C. to equilibrate. One ml/minute of the headspace was drawn for one minute through a Tenax™ headspace trap. The trap was removed and analyzed to determine the initial concentration of the malodor. The glass stopper was removed from the container and the test component and the malodor were left in contact for 60 minutes at 25° C. One ml/minute of the headspace was drawn for one minute through a Tenax™ headspace trap to determine the amount of malodor remaining. The Tenax traps were thermally desorbed into an Agilent 6890 GC/MS to analyze the quantity of hexyl amine. The results are shown in Table 3 below:

TABLE 3
Percent reduction of hexyl amine.

| | | % Reduction |
|---|---|---|
| | compound/compound mixture | of hexyl amine |
| 1. | citronellyl methylcrotonate | 53.6 |
| 2. | 50% compound 4 | 61.8 |
| | 50% citronellyl methylcrotonate | |
| 3. | benzyl cinnamate | 23.5 |
| 4. | 50% compound 4 | 61.4 |
| | 50% benzyl cinnamate | |
| 5. | Octyl methyl cinnamate | 54.1 |
| 6. | 50% compound 4 | 66.7 |
| | 50% octyl methyl cinnamate | |
| 7. | geranyl crotonate | 64.7 |
| 8. | 50% compound 4 | 78.4 |
| | 50% geranyl crotonate | |
| 9. | dihexyl fumarate | 68.7 |
| 10. | 50% compound 4 | 79.3 |
| | 50% dihexyl fumarate | |

As can be seen from the results above the use of compound 4, namely cyclopropanecarboxylic acid 2-[1-(3,3-dimethylcyclohexyl)ethoxy]-2-methylpropyl ester in combination with a class A compound significantly increases the effectiveness of the class A compound.

### EXAMPLE 2

### Determination of Malodor Reduction Potential of Different Compounds

Following the same procedure described in Example 1, compounds according to formula (B) were mixed with an equal amount of citronellyl methylcrotonate. 0.5 g of the resulting mixture was tested against 10 ul hexyl amine as a representative malodor. The results are given in Table 4, below.

TABLE 4
Percent reduction of hexyl amine

| | | % Reduction |
|---|---|---|
| | compound/compound mixture | of hexyl amine |
| 1. | citronellyl methylcrotonate | 61.3 |
| 2. | 50% compound 10 | 72.4 |
| | 50% citronellyl methylcrotonate | |
| 3. | 50% compound 1 | 64.4 |
| | 50% citronellyl methylcrotonate | |
| 4. | 50% compound C | 82.5 |
| | 50% citronellyl methylcrotonate | |
| 5. | 50% compound 7 | 72.1 |
| | 50% citronellyl methylcrotonate | |
| 6. | 50% compound 4 | 74.7 |
| | 50% citronellyl methylcrotonate | |

As can be seen from the results above the use of a compound of formula (B) in combination with a class A compound, namely citronellyl methylcrotonate, significantly increases the effectiveness of this known malodor counteractant molecule.

### EXAMPLE 3

### Determination of the Malodor Reduction Potential of a Compound Mixed with Known Malodor Counteractant Molecules

Following the same procedure described in Example 1,5-(1-(3,3-dimethyl-cyclohexyl)ethoxy)-5-methylhexan-2-one (compound 10) was mixed with an equal amount of compounds of formula (A). 0.5 g of the resulting mixture was tested against 10 ul hexyl amine as a representative malodor. The results are given in Table 5, below.

TABLE 5
Percent reduction of hexyl amine

| | | % Reduction |
|---|---|---|
| | compound/compound mixture | of hexyl amine |
| 1. | Geranyl crotonate | 71.0 |
| 2. | 50% compound 10 | 80.6 |
| | 50% geranyl crotonate | |
| 3. | Dihexyl fumarate | 78.1 |
| 4. | 50% compound 10 | 87.4 |
| | 50% dihexyl fumarate | |
| 5. | Benzyl cinnamate | 62.1 |
| 6. | 50% compound 10 | 71.5 |
| | 50% benzyl cinnamate | |
| 7. | Octyl methyl cinnamate | 58.7 |
| 8. | 50% compound 10 | 74.8 |
| | 50% octyl methyl cinnamate | |

### EXAMPLE 4

### Determination of the Malodor Reduction Potential of Different Mixtures

Test mixtures of a compound of formula (A) and a compound of formula (B) in the ratio as given in Table 6 (10% ethanol solution) of each was prepared. 0.5 g of a 1% ethanol solution of synthetic axilla malodor was placed onto cotton pads. 0.4 g of the ethanol solution of the test mixtures, respectively, was placed on each axilla malodor treated cotton pad. One pad was left untreated as the control. Each pad was allowed to dry for 15 minutes. The test cotton pads were randomized, and an expert panel of 5 was used to determine the intensity of the malodor. Each panelist was asked to check a box that represented the strength of the axilla malodor using a Labeled Magnitude Scale (LMS) (Barry G. Green, Pamela Dalton, Beverly Cowart, Greg Shaffer, Krystyna Rankin and Jennifer Higgins. Evaluating the Labeled Magnitude Scale for Measuring Sensations of Taste and Smell. Chemical Senses. Vol. 21, pp 323-334, 1996).

The % reduction of malodor was calculated by:

% Reduction=(Mean LMS Control−Mean LMS Test)/Mean LMS Control

TABLE 6
Percent reduction of axilla malodor

| | % Reduction |
|---|---|
| | of axilla malodor |
| 20% compound 4 | 71.0 |
| 80% geranyl crotonate | |
| 30% compound 4 | 71.3 |
| 70% geranyl crotonate | |
| 100% citronellyl methylcrotonate | 51.0 |
| 80% compound 10 | 71.3 |
| 20% citronellyl methylcrotonate | |
| 50% compound 10 | 67.4 |
| 50% citronellyl methylcrotonate | |
| 20% compound 10 | 58.2 |
| 80% citronellyl methylcrotonate | |

### EXAMPLE 5

A Givaudan fragrance composition containing solvent dipropylene glycol at 20% (Composition A) and a fragrance composition (Composition B) wherein 10% dipropylene glycol are replaced by 10% of a mixture of compound 4 and geranyl:crotonate (1:9); were compared for MOC efficacy using the sensory test described in Example 4. The results are shown in Table 7 below

TABLE 7
Reduction of axilla malodor

| | Composition A | Composition B |
|---|---|---|
| % reduction of malodor | 75.7 | 80.2 |

### EXAMPLE 6

### 5-[1′-(3″,3″-dimethylcyclohexyl)ethoxy]-5-methylhexan-2-one

Pyridinium chlorochromate (15.6 g, 72.4 mmol) was added in one dash to a vigorously stirred slurry of Celite® (16.0 g) in CH₂Cl₂ (200 mL). Within 20 min, a solution of 2-[1′-(3″,3″-dimethylcyclohexyl)ethoxy]-2-methylpropan-1-ol (15.0 g, 65.7 mmol), available according to WO 2002 096 852, in CH₂Cl₂ (200 mL) was added, and the resulting reaction mixture was stirred overnight at room temp., prior to filtration over a pad of silica gel and washing of the filter cake with CH₂Cl₂ (2×100 mL). The washings and the filtrate were combined, and the solvent removed in a rotary evaporator under reduced pressure. The residue obtained was purified by flash chromatography (silica gel; pentane/Et₂O, 19:1; R_{f}=0.63) to furnish 2-[1′-(3″,3″-dimethylcyclohexyl)ethoxy]-2-methylpropanal (10.0 g, 67%) as a colorless odoriferous liquid.

Under N₂ atmosphere, a solution of ethyl 2-(diethoxyphosphoryl)ethanoate (8.92 g, 39.8 mmol) in DME (10 mL) was added drop wise at room temp. to a stirred suspension of 95% NaH (1.00 g, 39.4 mmol) in DME (40 mL), upon which the temperature rose to 45° C. The reaction mixture was heated to reflux for 15 min prior to the drop wise addition of a solution of 2-[1′-(3″,3″-dimethylcyclohexyl)ethoxy]-2-methylpropanal (9.00 g, 39.8 mmol) in DME (20 mL) during 30 min. After further refluxing overnight, the reaction mixture was allowed to cool to room temp., and poured upon ice (400 g). After acidifying by addition of AcOH (2 mL, 34.9 mmol), the product was extracted with Et₂O (3×400 mL). The combined ethereal extracts were washed with water (400 mL) and brine (200 mL), dried (Na₂SO₄), and concentrated under reduced pressure in a rotary evaporator. The resulting residue (11.5 g) was purified by flash chromatography (silica gel; pentane/Et₂O, 19:1; R_{f}=0.41) to afford ethyl 4-[1′-(3″,3″-dimethylcyclohexyl)-ethoxy]-4-methylpent-2-enoate (10.9 g, 92%) as a colorless liquid. The thus prepared ethyl 4-[1′-(3″,3″-dimethylcyclohexyl)ethoxy]-4-methylpent-2-enoate (8.50 g, 28.7 mmol) was taken up in EtOH/water (1:1, 150 mL), and after addition of NaOH pellets (5.74 g, 143.6 mmol) with stirring, the resulting reaction mixture was refluxed overnight. Then the EtOH was removed in a rotary evaporator under reduced pressure, and the resulting residue diluted with water (500 mL) and washed with Et₂O (2×500 mL). The washings were discarded, the aqueous layer acidified with conc. aq. H₃PO₄, and then extracted with Et₂O (3×500 mL). The combined organic extracts were dried (Na₂SO₄) and evaporated to dryness in a rotary evaporator under reduced pressure to furnish 4-[1′-(3″,3″-dimethylcyclohexyl)-5-[1′-(3″,3″-dimethylcyclohexyl)-ethoxy]-5-methylhex-3-en-2-one (600 mg, 2.25 mmol) ethoxy]-4-methylpent-2-enoic acid (7.54 g, 98%).

At 0° C. under N₂ atmosphere, a solution of MeLi in Et₂O (1.6 M, 14.0 mL, 22.4 mL) was added drop wise to a stirred solution of 4-[1′-(3″,3″-dimethylcyclohexyl)-ethoxy]-4-methylpent-2-enoic acid (2.40 g, 8.94 mmol) in Et₂O (45 mL). After complete addition, the cooling bath was removed, and the reaction mixture heated to reflux for 2 h with stirring. GC control then indicated complete conversion, and thus the heating source was removed and replaced again by a cooling bath. The reaction mixture was quenched at 0° C. by addition of aq. HCl (5 N, 20 mL), and then diluted with water (100 mL). The product was extracted with Et₂O (2×300 mL), and the combined ethereal extracts were washed with water (200 mL) and brine (100 mL), prior to removal of the solvent in a rotary evaporator under reduced pressure. The resulting residue (2.25 g) was purified by flash chromatography (silica gel; pentane/Et₂O, 19:1; R_{f}=0.25) to provide 5-[1′-(3″,3″-dimethylcyclohexyl)ethoxy]-5-methylhex-3-en-2-one (1.25 g, 53%) as a colorless odoriferous liquid.

A suspension of 5-[1′-(3″,3″-dimethylcyclohexyl)ethoxy]-5-methylhex-3-en-2-one (600 mg, 2.25 mmol) and 10% Pd/C (100 mg, 0.09 mmol) in EtOAc (10 mL) was evacuated twice, and flushed with N₂. Following two cycles of flushing and evacuating with H₂, the reaction mixture was stirred at room temperature overnight under a positive pressure of H₂. After complete conversion was detected by GC, the reaction flask was evacuated and flushed with N₂, prior to removal of the catalyst by vacuum filtration over a pad of Celite®. The filter cake was washed with EtOAc (2×25 mL), and the washings were combined with the filtrate and concentrated under reduced pressure in a rotary evaporator. The resulting residue (570 mg) was purified by bulb-to-bulb distillation to provide at 85-100° C./0.05 mbar the title compound (480 mg, 84%) as a colorless odoriferous liquid.

IR (ATR): ν=1718 (s, νC═O), 1364/1383 (s, δCH₃), 1072 (s, νₛC—O—C), 1155/1135 (m, νₐₛC—O—C), 1461 (m, δC—H) cm⁻¹. ¹H NMR (CDCl₃): δ=0.70-0.85 (m, 1H, 2″-H_{b}), 0.86/0.87/0.89/0.90 (4s, 6H, 3″-Me₂), 1.01/1.02 (2d, J=6.5 Hz, 3H, 2′-H₃), 1.10/1.13 (2s, 6H, 5-Me₂), 1.32-1.67 (m, 8H, 1″-H, 2″-Ha, 4″-H₂-6″-H₂), 1.72 (t, J=7.0 Hz, 2H, 4-H₂), 2.16 (s, 3H, 1-H₃), 2.53 (t, J=7.0 Hz, 2H, 3-H₂), 3.32/3.33 (2quint, J=6.5 Hz, 1H, 1′-H). ¹³C NMR (CDCl₃): δ=19.2/19.4 (2q, C-2′), 22.2/22.3 (2t, C-5″), 24.5/24.6 (2q, 3″-Meₐₓ), 25.6/25.7/26.1/26.2 (4q, 5-Me₂), 27.9/29.2 (2t, C-6″), 29.8/29.9 (2q, C-1), 30.5/30.6 (2s, C-3″), 33.5/33.6 (2q, 3″-Me_{eq}), 36.0/36.1 (2t, C-4), 38.6/38.7 (2t, C-4″), 39.2/39.3 (2t, C-3), 40.2/40.3 (2d, C-1″), 41.3/42.2 (2t, C-2″), 70.8/70.9 (2d, C-1′), 73.7/73.8 (2s, C-5), 209.2/209.3 (2s, C-2). MS (70 eV): m/z (%)=157 (2) [C₁₀H₂₁O⁺], 139 (5) [C₁₀H₂₁O⁺—H₂O], 123 (8) [C₉H₁₅⁺], 113 (100) [C₇H₁₃O⁺], 97 (24) [C₁₀H₂₁O⁺—H₂O—C₃H₆], 69 (38) [C₅H₉⁺], 55 (28) [C₄H₇⁺], 43 (87) [C₃H₅⁺].

Odor description: Musky-rosy note with a fruity, pear-type nuance and some reminiscence to geranyl acetate.

1. A composition comprising
a. at least one compound of formula (A) 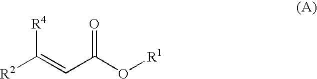
wherein
R¹ is linear or branched C₁-C₁₂ alkyl, linear or branched C₃-C₁₂ alkenyl, C₆-C₁₁ aryl, C₇-C₁₂ arylalkyl, or C₇-C₁₂ arylalkyl substituted with at least one O or N atom;
R² is linear or branched C₁-C₁₀ alkyl, C₆-C₁₀ aryl, C₇-C₁₀ alkoxyaryl, C₄-C₁₀ alkoxycarbonyl;
R⁴ is hydrogen or methyl; and
R² and R⁴ are either in the E or Z configuration with respect to the ester group;
and
b. at least one compound of formula (B) 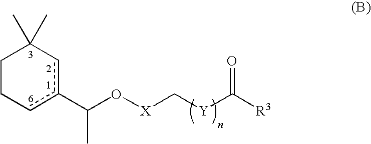
wherein
n is 0 or 1;
X is selected from the list of bivalent residues —C(CH₃)₂— and —C(O)—;
Y is selected from the list of bivalent residues —O—, —CH(CH₃)— and —CH₂—;
R³ is selected from the group consisting of C₁-C₃ alkyl, C₂-C₃ alkenyl, C₃-C₅ cycloalkyl, C₁-C₃ alkoxy, and NR′R″ wherein R′ and R″ are independently of each other hydrogen, methyl or ethyl; and
the bonds between C-1 and C-2, and C-1 and C-6 are single bonds; or one of the bonds between C-1 and C-2, and C-1 and C-6 together with the dotted line represents a double bond.

2. A composition according to claim 1 wherein the compound of formula (A) is selected from the group consisting of:
3,7-dimethyloct-6-enyl 3-methylbut-2-enoate,
geranyl crotonate,
dihexyl fumarate,
benzyl cinnamate,
phenyl cinnamate and
2-ethyl-hexyl-para-methoxy-cinnamate.

3. A composition according to claim 1 wherein the compound of formula (B) is selected from compounds wherein:
n is 0,
the bonds between C-1 and C-2, and C-1 and C-6 are single bonds, and wherein when
X=—C(CH₃)₂— and R³=methyl; or
X=—C(O)— and R³=methyl; or
X=—C(O)— and R³=—O—C₂H₅; or
X=—C(CH₃)₂— and R³=—O—C₂H₅; or
X=—C(CH₃)₂— and R³=ethyl.

4. A composition according to claim 1 wherein the compound of formula (B) is selected from compounds wherein:
n is 1,
the bonds between C-1 and C-2, and C-1 and C-6 are single bonds, and wherein when
X=—C(CH₃)₂—, Y=—O— and R³=ethyl; or
X=—C(O)—, Y=—O— and R³=ethyl; or
X=—C(CH₃)₂—, Y=—O— and R³=cyclopropyl; or
X=—C(O)—, Y=—O— and R³=cyclopropyl; or
X=—C(CH₃)₂—, Y=—O— and R³=vinyl; or
X=—C(O)—, Y=—O— and R³=vinyl; or
X=—C(CH₃)₂—, Y=—CH₂— and R³=methyl; or
X=—C(O)—, Y=—CH₂— and R=methyl; or
X=—C(O)—, Y=—CH₂— and R³=methoxy; or
X=—C(O)—, Y=—CH(CH₃)— and R³=methyl; or
X=—C(O)—, Y=—CH(CH₃)— and R³=methoxy; or
X=—C(O)—, Y=—CH(CH₃)— and R=—N(CH₃)₂; or
X=—C(O)—, Y=—CH₂— and R³=ethoxy.

5. A composition according to claim 1 wherein the compound of formula (B) is selected from compounds wherein n is 1, the bond between C-1 and C-6 is a single bond, the bond between C-1 and C-2 together with the dotted line is a double bond, X is —C(CH₃)₂—, Y is —O— and R³ is ethyl, vinyl or cyclopropyl.

6. A composition according to claim 1, wherein the composition further comprises at least one odorant.

7. A consumer product comprising an effective amount of a composition according to claim 1.

8. A consumer product according to claim 7 wherein the consumer product is selected from the group consisting of household products, personal care products and cosmetics.

9. A method of removing malodor from the air or from surfaces, comprising the step of: supplying to the air or surface an effective amount of a composition according to claim 1.

10. A method of enhancing the malodor reduction properties of a consumer product comprising the step of: admixing to the product at least one compound of formula (A) and at least one compound of formula (B) as defined in claim 1.

11. A compound of formula (B′) 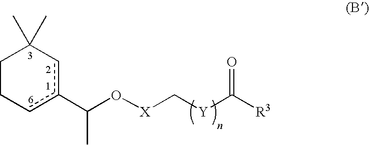
wherein
the bonds between C-1 and C-2, and C-1 and C-6 are single bonds; or one of the bonds between C-1 and C-2, and C-1 and C-6 together with the dotted line represents a double bond; and
I) n is 1;
X is selected from the list of bivalent residues —C(CH₃)₂— and —C(O)—;
Y is selected from the list of bivalent residues —CH(CH₃)— and —CH₂—; and
R³ is selected from the group consisting of CH₃, OCH₃, OC₂H₅ and N(CH₃)₂;
II) n is 0;
R³ is selected from the group consisting of C₁-C₃ alkyl, C₂-C₃ alkenyl, C₃-C₅ cycloalkyl, C₁-C₃ alkoxy, and NR′R″ wherein R′ and R″ are independently of each other hydrogen, methyl or ethyl; or
III) n is O;
X is —C(O)—; and
R³ is selected from the group consisting of C₂-C₃ alkyl, C₂-C₃ alkenyl, C₃-C₅ cycloalkyl, methoxy, and NR′R″ wherein R′ and R″ are independently of each other hydrogen, methyl or ethyl; with the proviso that 1-(3,3-dimethylcyclohexyl)ethyl methyl malonate is excluded.

12. A compound according to claim 11 wherein the compound of formula (B′) is selected from compounds wherein n is 0, the bonds between C-1 and C-2, and C-1 and C-6 are single bonds, and wherein when
X=—C(CH₃)₂— and R³=methyl; or
X=—C(CH₃)₂— and R³=—O—C₂H₅; or
X=—C(CH₃)₂— and R³=ethyl.

13. A compound according to claim 11 wherein the compound of formula (B′) is selected from compounds wherein n is 1, the bonds between C-1 and C-2, and C-1 and C-6 are single bonds, and wherein when
X=—C(CH₃)₂—, Y=—CH₂— and R³=methyl; or
X=—C(O)—, Y=—CH₂— and R³=methyl; or
X=—C(O)—, Y=—CH₂— and R³=methoxy; or
X=—C(O)—, Y=—CH(CH₃)— and R³=methyl; or
X=—C(O)—, Y=—CH(CH₃)— and R³=methoxy; or
X=—C(O)—, Y=—CH(CH₃)— and R³=—N(CH₃)₂; or
X=—C(O)—, Y=—CH₂— and R³=ethoxy.
